(19) United States
(12) Patent Application Publication
(10) Pub. No.: US 2025/0154081 A1
(43) Pub. Date: May 15, 2025

(54) HEAVY AROMATIC CONVERSION FOR INCREASED BTX PRODUCTION

(71) Applicant: UOP LLC, Des Plaines, IL (US)

(72) Inventors: Kristen E. Allaire, Chicago, IL (US); Stanley J. Frey, Madison, WI (US); David S. Lafyatis, Schaumburg, IL (US); Brett King, Prospect Heights, IL (US)

(21) Appl. No.: 18/806,332

(22) Filed: Aug. 15, 2024

Related U.S. Application Data
(60) Provisional application No. 63/599,017, filed on Nov. 15, 2023.

(51) Int. Cl. C07C 6/12

(52) U.S. Cl. CPC C07C 6/126; C07C 2523/28; C07C 2523/36; C07C 2523/42; C07C 2523/75; C07C 2523/755; C07C 2529/18

(57) ABSTRACT

The processes upgrade a low value heavy aromatics stream into a high value BTX precursor stream. It treats the naphthalenic molecules in the heavy aromatics stream comprising C₉ to C₁₂ aromatics prior to reaching the transalkylation reactor. The naphthalenic molecules are converted into BTX precursors by partially hydrogenating one of the aromatic rings and subsequently ring opening the saturated ring to form a monoaromatic prior to the transalkylation reactor. With naphthalene no longer present, the remaining C₁₁ monoaromatics may be processed in the aromatics complex for an overall increased BTX production.

### RELATED APPLICATIONS

This application claims priority to U.S. Provisional Patent Application Ser. No. 63/599,017, filed on Nov. 15, 2023, the entirety of which is incorporated herein by reference.

### BACKGROUND

C₁₁ and C₁₂ aromatic molecules are excellent p-xylene (pX) precursors due to their high methyl to phenyl ratio, on average. Presently, C₁₁ and C₁₂ aromatics are limited from aromatics complex feeds because they carry coke precursors (i.e., naphthalenic molecules). As a result, C₁₁ and C₁₂ molecules that would be precursors to Ci and C₁₂ aromatic formation in the naphtha reformer are usually sent to a lower value stream (e.g., kerosene, diesel fuel, jet fuel, and the like) rather than being converted to C₁₁ and C₁₂ aromatics in the naphtha reformer and further processed to benzene, toluene, and xylenes (BTX) in the aromatics complex. Alternatively, in cases where C₁₁ and C₁₂ molecules that would be precursors to C₁₁ and C₁₂ aromatic formation in the naphtha reformer are allowed in the reformer and are allowed in the aromatics complex, they are expelled from the heavy aromatics column bottom stream and do not contribute to BTX production.

This results in the loss of compounds that could be converted into BTX products.

Therefore, there is a need for a process that would allow the use of C₁₁ and C₁₂ aromatics in an aromatic complex thereby increasing the production of BTX products.

### BRIEF DESCRIPTION OF THE DRAWING

FIG. 1 is an illustration of a prior art aromatics complex having no heavy aromatics unit.

FIG. 2 is one embodiment of a complex with heavy aromatics unit.

### DESCRIPTION

The processes upgrade a low value heavy aromatics stream which would otherwise be expelled from the heavy aromatics column bottom stream into a high value BTX precursor stream. It treats the naphthalenic molecules, such as naphthalene and alkyl-naphthalenes, in the heavy aromatics stream comprising C₉ to C₁₂ aromatics prior to reaching a downstream transalkylation reactor. The naphthalenic molecules are converted into BTX precursors by partially hydrogenating one of the aromatic rings and subsequently ring opening the saturated ring to form a monoaromatic. With the naphthalenes content substantially reduced, the remaining C₁₁ and C₁₂ monoaromatics may be processed in the transalkylation reactor for an overall increased BTX production.

The process has a relatively low capital cost and operating cost and improves the molecular management of aromatics for BTX production. It provides the opportunity for reduced carbon intensity and increased net hydrogen, as well increasing the BTX production per ton of feed.

The process could be retrofit in existing aromatics complexes and designed into new aromatics complexes. It involves the addition of a reactor that would contain a catalyst with a hydrogenation function (such as cobalt and molybdenum on an alumina support) to hydrogenate one ring of the naphthalenic compounds to form tetralin and substituted tetralins. The reactor system may also contain a second catalyst bed with a catalyst with a cracking function (such as molybdenum on an MFI-type zeolite and alumina support) to ring open and then de-alkylate the tetralins to form single ring aromatic compounds. Alternatively, ring opening/de-alkylation may be performed in a separate reactor or in a downstream transalkylation reactor.

If the heavy aromatics stream also includes indane and/or alkyl indanes, the catalyst with the cracking function may also ring open the indanes and alkyl indanes, and dealkylate the ring-opened indanes and alkyl indanes.

The heavy aromatics conversion process takes place in an aromatics complex and involves the conversion of bicyclic aromatic compounds. The heavy aromatics stream that is fed into the heavy aromatics conversion process has 95% or more aromatic content and comprises C₉ to C₁₂ aromatics comprising naphthalene and alkyl naphthalenes. The naphthalene and the alkyl naphthalenes in the heavy aromatics stream are hydrogenated in the presence of a catalyst with a hydrogenation function under hydrogenation conditions to form a partially hydrogenated reaction mixture comprising tetralin and alkyl tetralin.

The partially hydrogenated reaction mixture may be further processed in the presence of a catalyst with ring opening and dealkylation functions under ring opening and dealkylation reaction conditions thereby ring opening the tetralin and alkyl tetralin forming alkyl monoaromatics, and dealkylating the alkyl monoaromatics to form dealkylated monoaromatics.

The naphthalene and the alkyl naphthalene can be hydrogenated in a first reactor, and the partially hydrogenated reaction mixture processing can take place in a second reactor. Alternatively, the hydrogenation of the naphthalene and the alkyl naphthalene and the processing of the partially hydrogenated reaction mixture can take place in a single reactor.

The process may also include transalkylating the dealkylated monoaromatics in the presence of an aromatic transalkylation catalyst under aromatic transalkylation reactions conditions to form a transalkylated product.

In some embodiments, hydrogenating the naphthalene and the alkyl naphthalene and processing the partially hydrogenated reaction mixture take place in different beds in a single reactor.

In some embodiments, hydrogenating the naphthalene and the alkyl naphthalene and processing the partially hydrogenated reaction mixture take place in different beds in a first reactor, and transalkylating the dealkylated monoaromatics takes place in a second reactor.

In some embodiments, hydrogenating the naphthalene and the alkyl naphthalene takes place in a first reactor, and processing the partially hydrogenated reaction mixture and transalkylating the dealkylated monoaromatics take place in different beds in a second reactor.

In some embodiments, hydrogenating the naphthalene and the alkyl naphthalene takes place in a first reactor, processing the partially hydrogenated reaction mixture takes place in a second reactor, and transalkylating the dealkylated monoaromatics takes place in a third reactor.

In some embodiments, hydrogenating the naphthalene and the alkyl naphthalene, processing the partially hydrogenated reaction mixture, and transalkylating the dealkylated monoaromatics take place in different beds in a single reactor.

In some embodiments, hydrogenating the naphthalene and the alkyl naphthalene takes place in a first reactor, and processing the partially hydrogenated reaction mixture and transalkylating the dealkylated monoaromatics take place in a second reactor, and the aromatic transalkylation catalyst has the ring opening and dealkylation functions.

In some embodiments, hydrogenating the naphthalene and the alkyl naphthalene takes place in a first bed in a reactor, and processing the partially hydrogenated reaction mixture and transalkylating the dealkylated monoaromatics take place in a second bed in the reactor, and the aromatic transalkylation catalyst has the ring opening and dealkylation functions.

In some embodiments, the heavy aromatics stream further comprises indane and alkyl indane, and processing the partially hydrogenated reaction mixture comprises ring opening the indane and alkyl indane forming additional monoaromatics, and dealkylating the additional alkyl monoaromatics to form additional dealkylated monoaromatics.

In some embodiments, the heavy aromatics stream further comprises C₆ to C₈ aromatics and C₁₃ to C₁₄ aromatics. In some embodiments, C₆-C₈ aromatics could be added downstream of the heavy aromatics conversion process.

In some embodiments, the catalyst with the hydrogenation function comprises a metal from Groups 6-10 of the Periodic Table. In some embodiments, the catalyst with the hydrogenation function comprises Mo, Ni, Co or combinations thereof.

In some embodiments, the catalyst with the ring opening and dealkylation function comprises a metal from Groups 6-10 and a zeolite, the zeolite comprising a MFI Type zeolite or a MOR Type zeolite, or combination thereof.

In some embodiments, the catalyst with the ring opening and dealkylation function comprises Mo, Pt, or Re, or combination thereof.

In some embodiments, the hydrogenation conditions comprise a temperature in a range of 200° C. to 450° C., or a pressure in a range of 1 MPa to 5 MPa, or combinations thereof.

In some embodiments, the ring opening and dealkylation conditions comprise a temperature in a range of 200° C. to 450° C., or a pressure in a range of 1 MPa to 5 MPa, or combinations thereof.

In some embodiments, the aromatic transalkylation conditions comprise a temperature in a range of 200° C. to 450° C., or a pressure in a range of 1 MPa to 5 MPa, or combinations thereof.

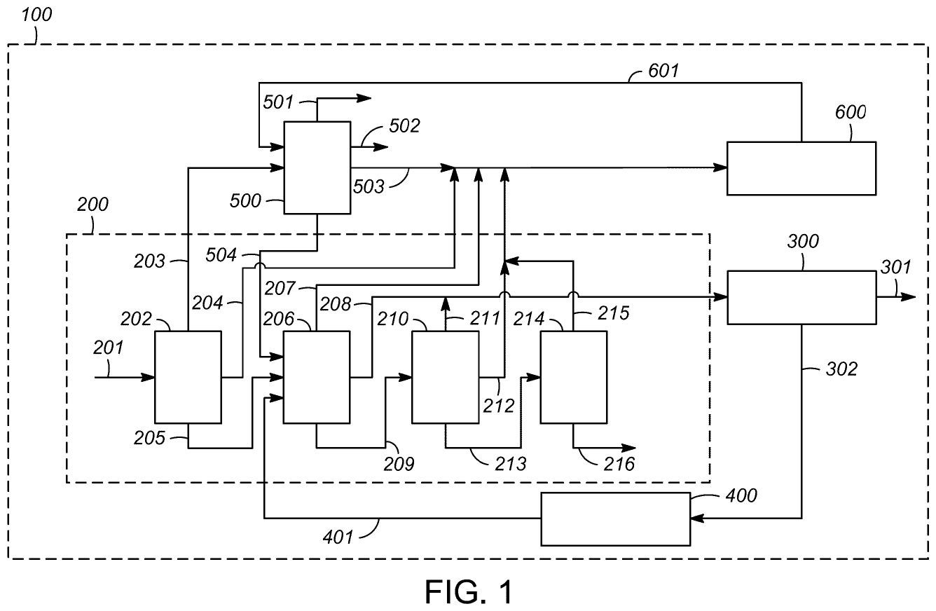

The heavy aromatics stream is typically obtained from a naphtha reformer or a variety of other sources, e.g. petroleum refining, thermal or catalytic cracking, or petrochemical conversions with suitably rich aromatic content. Processing of the aromatic feed to produce para-xylene within the aromatics complex 100 according to the known art would typically proceed as shown in FIG. 1. The aromatics complex 100 comprises a xylene fractionation section 200, an adsorptive separation section 300, an isomerization section 400, a benzene/toluene separation section 500, and a transalkylation reaction section 600.

The feed stream 201, for example but without limiting the invention, will contain heavy aromatics, e.g., C₉ and heavier alkyl monoaromatics, naphthalene, and alkyl naphthalenes, in a mixture of non-aromatics, benzene, toluene, C₈ alkyl monoaromatics isomers, and mixtures thereof comprised of C₅-C₁₁ and heavier hydrocarbons. The present invention is demonstrated by performance with a feed stream containing a substantial content, e.g., about 1 wt-% or more, or at least 5 wt-%, of C₉ and heavier hydrocarbons.

The xylene fractionation section 200 comprises a reformate splitter fractionator 202, an aromatic “stripper” fractionator 206, an aromatic “rerun” fractionator 210, and a “heavy aromatics” fractionator 214.

The feed stream from 201 is separated in the reformate splitter fractionator 202 into a reformate splitter overhead stream 203 comprising mainly C₅-C₇ non-aromatics, benzene and toluene, a reformate splitter side stream 204 comprising mainly benzene and toluene, and a reformate splitter bottom stream 205 comprising mainly C₈ and heavier aromatics.

Reformate splitter bottom stream 205, C₈ alkyl aromatic recycle stream 504 from the benzene/toluene separation section 500 comprising mainly benzene and toluene, and isomerization effluent stream 401 from the isomerization section 400 are combined and separated in an aromatic stripper fractionator 206 into an aromatic stripper overhead stream 207 comprising mainly benzene and toluene, an aromatic stripper side stream 208 comprising mainly C₈ alkyl aromatics, and an aromatic stripper bottom stream 209 comprising mainly a concentrated mixture of C₈ and heavier alkyl monoaroamatics, naphthalene, and alkyl naphthalenenes.

The aromatic stripper bottom stream 209 from the aromatic stripper fractionator 206 is separated in an aromatic “rerun” fractionator 210 into an aromatic rerun overhead stream 211 comprising mainly of C₈ alkyl monoaromatics, an aromatic rerun side stream 212 comprising mainly C₉ alkyl monoaromatics, and an aromatic rerun bottom stream 213 comprising mainly C₁₀ and heavier alkyl monoaromatics and concentrated naphthalenes, and alkyl naphthalenes.

The aromatic rerun bottom stream 213 is separated in a “heavy aromatics” fractionator 214 into a heavy aromatics overhead stream 215, comprising C₉ to C₁₁ aromatics and potentially comprising naphthalene and alkyl naphthalenes, and a heavy aromatics bottom stream 216 comprising mainly C₁₁ and heavier hydrocarbons. In the aromatics complex of FIG. 1, the amount of naphthalene and alkyl naphthalenes in the heavy aromatics overhead stream 215 must be limited such that the combined feed to section 600 is, e.g., less than 0.5 wt-%, or at most 2.0 wt-% naphthalenes and alkyl naphthalenes, to prevent these components from having an adverse impact on the downstream transalkylation reaction section 600, isomerization section 400, adsorptive separation section 300, and benzene/toluene separation section 500. These components rejected to the heavy aromatics bottoms stream 216 represent a loss in potential BTX precursors.

The reformate splitter overhead stream 203 is sent to the benzene/toluene section 500 forming a benzene stream 501, a raffinate stream 502, a toluene stream 503, and a C8 alkyl aromatics stream 504. The C8 alkyl aromatics stream 504 is sent to the aromatic stripper fractionator 206 as recycle.

The reformate splitter side stream 204, the aromatic stripper overhead stream 207, the aromatic rerun side stream 212, and the toluene stream 503 are combined and sent to the transalkylation section 600. The transalkylation effluent stream 601 is recycled to the benzene/toluene section 500.

The aromatic stripper side stream 208 is combined with the aromatic rerun overhead stream 211 as a mixture of xylene isomers. The adsorptive separation section 300 comprises a p-xylene selective embodiment to form a p-xylene rich stream 301, and a mixed xylene stream 302. In other embodiments, the desired xylene isomer can also be m-xylene or o-xylene.

The mixed xylene stream 302 is sent to the isomerization section 400. The isomerization effluent stream 401 is recycled to the aromatic stripper fractionator 206.

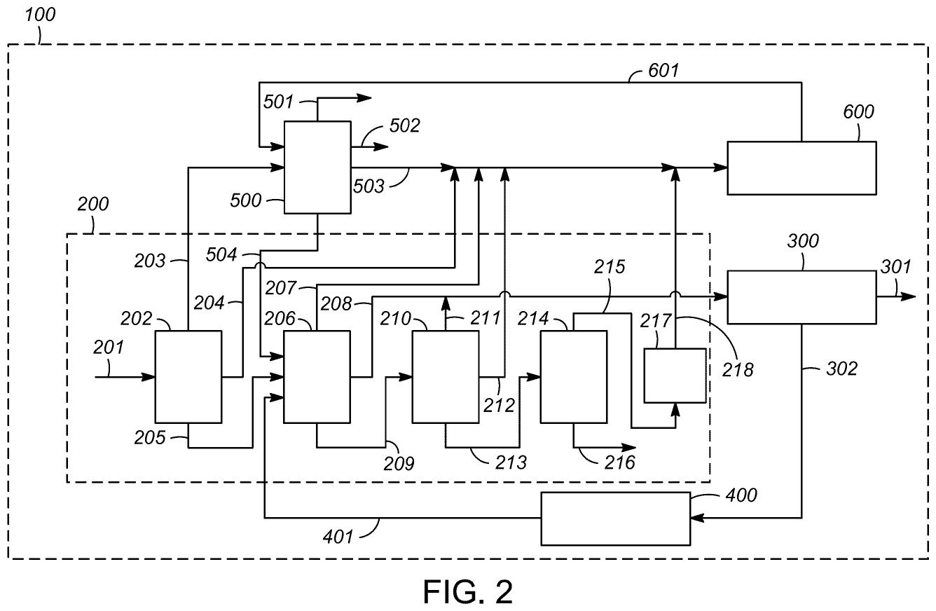

FIG. 2 illustrates one embodiment of a process according to the present invention. This scheme utilizes the aromatics complex 100 depicted in FIG. 1 with the addition of the heavy aromatics processing unit 217 within the xylene fractionation section 200. In this embodiment shown in FIG. 2, the heavy aromatics overhead stream 215 is routed through the heavy aromatics conversion unit 217, rather than to the feed of the transalkylation section 600 as was illustrated in FIG. 1. In FIG. 2, the heavy aromatics overhead stream 215 comprising naphthalene and alkyl naphthalene is directed to the heavy aromatics unit 217 where the partial hydrogenation to tetralin and substituted tetralins takes place, and in some cases, the ring opening and dealkylation reactions take place. Therefore, the amount of naphthalene and alkyl naphthalenes in the heavy aromatics overhead stream 215 need not be limited as in the process of FIG. 1, and these naphthalene and alkyl naphthalenes components contribute to increased BTX production from the aromatics complex, rather than being rejected from the aromatics complex in the heavy aromatics bottom stream 216. The heavy aromatics unit effluent stream 218 from the heavy aromatics unit 217 is combined with the reformate splitter side stream 204, the aromatic stripper overhead stream 207, the aromatic rerun side stream 212, and the toluene stream 503 and sent to the transalkylation section 600.

The exemplary flow scheme embodies one example of an aromatics complex 100 known in the art. It should be understood that numerous other examples of aromatics complexes exist, and the exemplary embodiments are not intended to limit the scope, application, or configuration of the application in any way. For example, additional feed streams from outside of the FIG. 2 aromatics complex could be introduced to the FIG. 2 aromatics complex to pass through the heavy aromatics conversion unit 217 and enhance overall BTX production.

The embodiments represent an outline for those skilled in the art to implement the process, considering that changes may be made in the function and arrangement of elements described in the exemplary embodiment without departing from the scope, as described in the specific claims.

### EXAMPLES

### Example 1

A hydrogenation catalyst comprising alumina, cobalt, and molybdenum was prepared for pilot plant testing by the extrusion forming process.

The catalyst was tested in a single bed reactor to the evaluate naphthalene and methyl naphthalene conversion to tetralin and alkyl tetralin at 220-300° C., 400 psig, 1 H₂/HC, 3 WHSV, and with the 100% aromatic content feed described in Table 1.

TABLE 1

| Feed | Wt % |
|---|---|
| Naphthalene | 2.3 |
| 1-methylnaphthalene | 0.7 |
| 2-methylnaphthalene | 0.2 |
| Mesitylene | 96.8 |

As shown in Table 2, naphthalene and methylnaphthalene hydrogenate to form tetralin and methyltetralin with minimal monoaromatic ring loss at the given conditions and temperature range.

TABLE 2

| Temperature, ° C. | 220 | 250 | 270 | 300 |
|---|---|---|---|---|
| Naphthalene Yield, wt % | 1.6 | 1.1 | 0.6 | 0.2 |
| Methylnaphthalene Yield, wt % | 0.7 | 0.5 | 0.2 | 0.1 |
| Tetralin Yield, wt % | 0.6 | 1.2 | 1.7 | 2.1 |
| Methyltetralin Yield, wt % | 0.8 | 0.5 | 0.8 | 0.9 |
| Monoaromatic ring loss, mol % | 0.06 | 0.14 | 0.30 | 0.39 |

### Example 2

A catalyst with ring opening and dealkylation functions containing Mo, MFI Zeolite and MOR zeolite was prepared for pilot plant testing by the extrusion forming process.

The catalyst was tested in a single bed reactor to the evaluate tetralin and indane conversion at 400 psig, 3 H₂/HC, 3 WHSV, and with the 100% aromatic content feed described in Table 3.

TABLE 3

| Feed | Wt % |
|---|---|
| Tetralin | 4 |
| Indane | 2 |
| Toluene | 68 |
| Mesitylene | 26 |

As shown in Table 3, tetralin and indane are converted at the given conditions and temperature range.

TABLE 4

| Temperature, ° C. | 328 | 336 | 343 |
|---|---|---|---|
| Tetralin Conversion | 94% | 96% | 97% |
| Indane Conversion | 22% | 38% | 58% |

### Specific Embodiments

While the following is described in conjunction with specific embodiments, it will be understood that this description is intended to illustrate and not limit the scope of the preceding description and the appended claims.

A first embodiment of the invention is a heavy aromatics conversion process located in an aromatics complex for converting bicyclic aromatic compounds comprising providing a heavy aromatics stream having 95% or more aromatic content and comprising C₉ to C₁₁ aromatics comprising naphthalene and alkyl naphthalene; hydrogenating the naphthalene and the alkyl naphthalene in the presence of a catalyst with a hydrogenation function under hydrogenation conditions to form a partially hydrogenated reaction mixture comprising tetralin and alkyl tetralin. An embodiment of the invention is one, any or all of prior embodiments in this paragraph up through the first embodiment in this paragraph further comprising processing the partially hydrogenated reaction mixture in the presence of a catalyst with ring opening and dealkylation functions under ring opening and dealkylation reaction conditions thereby ring opening the tetralin and alkyl tetralin forming alkyl monoaromatics, and dealkylating the alkyl monoaromatics to form dealkylated monoaromatics. An embodiment of the invention is one, any or all of prior embodiments in this paragraph up through the first embodiment in this paragraph further comprising transalkylating the dealkylated monoaromatics in the presence of an aromatic transalkylation catalyst under aromatic transalkylation reactions conditions to form a transalkylated product. An embodiment of the invention is one, any or all of prior embodiments in this paragraph up through the first embodiment in this paragraph wherein hydrogenating the naphthalene and the alkyl naphthalene and processing the partially hydrogenated reaction mixture take place in different beds in a single reactor. An embodiment of the invention is one, any or all of prior embodiments in this paragraph up through the first embodiment in this paragraph wherein hydrogenating the naphthalene and the alkyl naphthalene and processing the partially hydrogenated reaction mixture take place in different beds in a first reactor, and wherein transalkylating the dealkylated monoaromatics takes place in a second reactor. An embodiment of the invention is one, any or all of prior embodiments in this paragraph up through the first embodiment in this paragraph wherein hydrogenating the naphthalene and the alkyl naphthalene takes place in a first reactor, and wherein processing the partially hydrogenated reaction mixture and transalkylating the dealkylated monoaromatics take place in different beds in a second reactor. An embodiment of the invention is one, any or all of prior embodiments in this paragraph up through the first embodiment in this paragraph wherein hydrogenating the naphthalene and the alkyl naphthalene takes place in a first reactor, wherein processing the partially hydrogenated reaction mixture takes place in a second reactor, and wherein transalkylating the dealkylated monoaromatics takes place in a third reactor. An embodiment of the invention is one, any or all of prior embodiments in this paragraph up through the first embodiment in this paragraph wherein hydrogenating the naphthalene and the alkyl naphthalene, processing the partially hydrogenated reaction mixture, and transalkylating the dealkylated monoaromatics take place in different beds in a single reactor. An embodiment of the invention is one, any or all of prior embodiments in this paragraph up through the first embodiment in this paragraph wherein hydrogenating the naphthalene and the alkyl naphthalene takes place in a first reactor, and wherein processing the partially hydrogenated reaction mixture and transalkylating the dealkylated monoaromatics take place in a second reactor, and wherein the aromatic transalkylation catalyst has the ring opening and dealkylation functions. An embodiment of the invention is one, any or all of prior embodiments in this paragraph up through the first embodiment in this paragraph wherein hydrogenating the naphthalene and the alkyl naphthalene takes place in a first bed in a reactor, and wherein processing the partially hydrogenated reaction mixture and transalkylating the dealkylated monoaromatics take place in a second bed in the reactor, and wherein the aromatic transalkylation catalyst has the ring opening and dealkylation functions. An embodiment of the invention is one, any or all of prior embodiments in this paragraph up through the first embodiment in this paragraph wherein the heavy aromatics stream further comprises indane and alkyl indane and wherein processing the partially hydrogenated reaction mixture comprises processing ring opening the indane and alkyl indane, forming additional monoaromatics, and dealkylating the additional alkyl monoaromatics to form additional dealkylated monoaromatics. An embodiment of the invention is one, any or all of prior embodiments in this paragraph up through the first embodiment in this paragraph wherein the heavy aromatics stream further comprises C6 to C8 aromatics and C12 to C13 aromatics. An embodiment of the invention is one, any or all of prior embodiments in this paragraph up through the first embodiment in this paragraph wherein the catalyst with the hydrogenation function comprises a metal from Groups 6-10 of the Periodic Table. An embodiment of the invention is one, any or all of prior embodiments in this paragraph up through the first embodiment in this paragraph wherein the catalyst with the hydrogenation function comprises Mo, Ni, Co or combinations thereof. An embodiment of the invention is one, any or all of prior embodiments in this paragraph up through the first embodiment in this paragraph wherein the catalyst with the ring opening and dealkylation function comprises a metal from Groups 6-10 and a zeolite, the zeolite comprising a MFI Type zeolite or a MOR Type zeolite, or combination thereof. An embodiment of the invention is one, any or all of prior embodiments in this paragraph up through the first embodiment in this paragraph wherein the catalyst with the ring opening and dealkylation function comprises Mo, Pt, or Re, or combination thereof. An embodiment of the invention is one, any or all of prior embodiments in this paragraph up through the first embodiment in this paragraph wherein the hydrogenation conditions comprise a temperature in a range of 200° C. to 450° C., or a pressure in a range of 1 MPa to 5 MPa, or combinations thereof. An embodiment of the invention is one, any or all of prior embodiments in this paragraph up through the first embodiment in this paragraph wherein the ring opening and dealkylation conditions comprise a temperature in a range of 200° C. to 450° C., or a pressure in a range of 1 MPa to 5 MPa, or combinations thereof. An embodiment of the invention is one, any or all of prior embodiments in this paragraph up through the first embodiment in this paragraph wherein the aromatic transalkylation conditions comprise a temperature in a range of 200° C. to 450° C., or a pressure in a range of 1 MPa to 5 MPa, or combinations thereof. An embodiment of the invention is one, any or all of prior embodiments in this paragraph up through the first embodiment in this paragraph wherein hydrogenating the naphthalene and the alkyl naphthalene and processing the partially hydrogenated reaction mixture take place in a separate reactor.

A second embodiment of the invention is a heavy aromatics conversion process located in an aromatics complex for converting bicyclic aromatic compounds comprising providing a heavy aromatics stream having 95% or more aromatic content and comprising C₉ to C₁₁ aromatics comprising naphthalene and alkyl naphthalene; hydrogenating the naphthalene and the alkyl naphthalene in the presence of a catalyst with a hydrogenation function under hydrogenation conditions to form a partially hydrogenated reaction mixture comprising tetralin and alkyl tetralin; processing the partially hydrogenated reaction mixture in the presence of a catalyst with ring opening and dealkylation functions under ring opening and dealkylation reaction conditions thereby ring opening the tetralin and alkyl tetralin forming alkyl monoaromatics, and dealkylating the alkyl monoaromatics to form dealkylated monoaromatics; and transalkylating the dealkylated monoaromatics in the presence of an aromatic transalkylation catalyst under aromatic transalkylation reactions conditions to form a transalkylated product.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. A heavy aromatics conversion process located in an aromatics complex for converting bicyclic aromatic compounds comprising:
providing a heavy aromatics stream having 95% or more aromatic content and comprising C₉ to C₁₁ aromatics comprising naphthalene and alkyl naphthalene; and
hydrogenating the naphthalene and the alkyl naphthalene in the presence of a catalyst with a hydrogenation function under hydrogenation conditions to form a partially hydrogenated reaction mixture comprising tetralin and alkyl tetralin.

2. The process of claim 1 further comprising:
processing the partially hydrogenated reaction mixture in the presence of a catalyst with ring opening and dealkylation functions under ring opening and dealkylation reaction conditions thereby ring opening the tetralin and alkyl tetralin forming alkyl monoaromatics, and dealkylating the alkyl monoaromatics to form dealkylated monoaromatics.

3. The process of claim 2 further comprising:
transalkylating the dealkylated monoaromatics in the presence of an aromatic transalkylation catalyst under aromatic transalkylation reactions conditions to form a transalkylated product.

4. The process of claim 2 wherein hydrogenating the naphthalene and the alkyl naphthalene and processing the partially hydrogenated reaction mixture take place in different beds in a single reactor.

5. The process of claim 3 wherein hydrogenating the naphthalene and the alkyl naphthalene and processing the partially hydrogenated reaction mixture take place in different beds in a first reactor, and wherein transalkylating the dealkylated monoaromatics takes place in a second reactor.

6. The process of claim 3 wherein hydrogenating the naphthalene and the alkyl naphthalene takes place in a first reactor, and wherein processing the partially hydrogenated reaction mixture and transalkylating the dealkylated monoaromatics take place in different beds in a second reactor.

7. The process of claim 3 wherein hydrogenating the naphthalene and the alkyl naphthalene takes place in a first reactor, wherein processing the partially hydrogenated reaction mixture takes place in a second reactor, and wherein transalkylating the dealkylated monoaromatics takes place in a third reactor.

8. The process of claim 3 wherein hydrogenating the naphthalene and the alkyl naphthalene, processing the partially hydrogenated reaction mixture, and transalkylating the dealkylated monoaromatics take place in different beds in a single reactor.

9. The process of claim 3 wherein hydrogenating the naphthalene and the alkyl naphthalene takes place in a first reactor, and wherein processing the partially hydrogenated reaction mixture and transalkylating the dealkylated monoaromatics take place in a second reactor, and wherein the aromatic transalkylation catalyst has the ring opening and dealkylation functions.

10. The process of claim 3 wherein hydrogenating the naphthalene and the alkyl naphthalene takes place in a first bed in a reactor, and wherein processing the partially hydrogenated reaction mixture and transalkylating the dealkylated monoaromatics take place in a second bed in the reactor, and wherein the aromatic transalkylation catalyst has the ring opening and dealkylation functions.

11. The process of claim 2 wherein the heavy aromatics stream further comprises indane and alkyl indane and wherein processing the partially hydrogenated reaction mixture comprises processing ring opening the indane and alkyl indane, forming additional monoaromatics, and dealkylating the additional alkyl monoaromatics to form additional dealkylated monoaromatics.

12. The process of claim 1 wherein the heavy aromatics stream further comprises C₆ to C₈ aromatics and C₁₂ to C₁₃ aromatics.

13. The process of claim 1 wherein the catalyst with the hydrogenation function comprises a metal from Groups 6-10 of the Periodic Table.

14. The process of claim 13 wherein the catalyst with the hydrogenation function comprises Mo, Ni, Co or combinations thereof.

15. The process of claim 2 wherein the catalyst with the ring opening and dealkylation function comprises a metal from Groups 6-10 and a zeolite, the zeolite comprising a MFI Type zeolite or a MOR Type zeolite, or combination thereof.

16. The process of claim 15 wherein the catalyst with the ring opening and dealkylation function comprises Mo, Pt, or Re, or combination thereof.

17. The process of claim 1 wherein the hydrogenation conditions comprise a temperature in a range of 200° C. to 450° C., or a pressure in a range of 1 MPa to 5 MPa, or combinations thereof.

18. The process of claim 2 wherein the ring opening and dealkylation conditions comprise a temperature in a range of 200° C. to 450° C., or a pressure in a range of 1 MPa to 5 MPa, or combinations thereof.

19. The process of claim 3 wherein the aromatic transalkylation conditions comprise a temperature in a range of 200° C. to 450° C., or a pressure in a range of 1 MPa to 5 MPa, or combinations thereof.

20. A heavy aromatics conversion process located in an aromatics complex for converting bicyclic aromatic compounds comprising:
providing a heavy aromatics stream having 95% or more aromatic content and comprising C₉ to C₁₁ aromatics comprising naphthalene and alkyl naphthalene; and
hydrogenating the naphthalene and the alkyl naphthalene in the presence of a catalyst with a hydrogenation function under hydrogenation conditions to form a partially hydrogenated reaction mixture comprising tetralin and alkyl tetralin;
processing the partially hydrogenated reaction mixture in the presence of a catalyst with ring opening and dealkylation functions under ring opening and dealkylation reaction conditions thereby ring opening the tetralin and alkyl tetralin forming alkyl monoaromatics, and dealkylating the alkyl monoaromatics to form dealkylated monoaromatics; and
transalkylating the dealkylated monoaromatics in the presence of an aromatic transalkylation catalyst under aromatic transalkylation reactions conditions to form a transalkylated product.
